(19) United States
(12) Patent Application Publication
(10) Pub. No.: US 2024/0305588 A1
(43) Pub. Date: Sep. 12, 2024

(54) USING MACHINE LEARNING TECHNIQUES TO ROUTE CONSUMER INTERACTIONS FROM AN AUTOMATED MODE OF COMMUNICATION TO A SECOND MODE OF COMMUNICATION

(71) Applicant: Providence St. Joseph Health, Seattle, WA (US)

(72) Inventors: Andy L. Chu, Redmond, WA (US); Wayne T. Foley, Seattle, WA (US)

(21) Appl. No.: 18/182,248

(22) Filed: Mar. 10, 2023

(51) Int. Cl. H04L 51/02; G06N 3/02; G16H 10/60

(52) U.S. Cl. CPC H04L 51/02; G06N 3/02; G16H 10/60

(57) ABSTRACT

A facility for automatically managing live interactions is described. In response to receiving a request for live interaction from a user, the facility causes an automatic live interaction to be conducted with the user in which messages are received from the user and sent to the user. The facility periodically uses an up-to-date textual transcript for the automatic live interaction to assess whether the live interaction is one well-suited to a human live interaction. In response to determining that it is, the facility causes a human live interaction to be initiated between the user and a human agent in place of the automatic live interaction, and causes to be presented to the human agent text corresponding to at least some of the messages sent from and to the user during the automatic live interaction.

### BACKGROUND

Some organizations have made it possible for consumers to use a variety of modes of communication to communicate with the organization. For example, some healthcare organizations permit a patient to exchange textual chat or text messages with a person or an intelligent agent, and also speak with a person or an intelligent agent, among other modes of communication.

### BRIEF DESCRIPTION OF THE DRAWINGS

FIG. 1 is a block diagram showing some of the components typically incorporated in at least some of the computer systems and other devices on which the facility operates.

FIG. 2 is a flow diagram showing a process performed by the facility in some embodiments in order to train one or more machine learning models used by the facility.

FIG. 3 is a data flow diagram showing the operation of the facility in some embodiments to route consumer interactions between communication modes.

FIG. 4 is a flow diagram showing a process performed by the facility in some embodiments to route consumer interactions between communication modes.

FIG. 5 is a display diagram showing sample contents of a first display presented by the facility in some embodiments to a human agent to whom an interaction is routed by the facility.

FIG. 6 is a display diagram showing sample contents of a second display presented by the facility in some embodiments to a human agent to whom an interaction is routed by the facility.

FIG. 7 is a display diagram showing sample contents of a third display presented by the facility in some embodiments to a human agent to whom an interaction is routed by the facility.

FIG. 8 is a display diagram showing a sample display presented by the facility to a consumer in some embodiments.

### DETAILED DESCRIPTION

The inventors have recognized that offering consumers a diverse set of communication modes can lead to confusion and frustration on the part of those consumers. First, it can be difficult to choose the best mode for a particular interaction, and successfully navigate to and through it. As one example, to speak with a human representative, it is often necessary to find one of several phone numbers that are associated with the organization that is for calling a human representative. It is then often necessary navigate to a human representative capable of addressing the consumer's particular concern via an automated-response system, by either (1) listening to and digesting a series of spoken menus, and pressing a particular phone key in order to select the correct response to each one; or (2) speaking a description of their reason for calling for automatic comparison to an undisclosed list of candidate subjects.

Next, it is often true that a particular interaction requires switching modes, such as from a text-based interaction with an automated agent to a spoken interaction with a human agent. This process can be difficult for the consumer, including determining that a mode switch should be made, determining how to accomplish the mode switch, and performing navigation to or within the new mode. Also, because of fragmentation among the individual systems used by an organization to support the different modes of communication, information provided by the consumer using the first mode of the interaction is often not available for use in the second mode, and must be repeated by the consumer. For example, a consumer may have authenticated in a first, text-based mode, and also provided information about an appointment they need to schedule; when this information is not available for use in a second, voice-based mode of the interaction, the consumer must repeat it by re-authenticating and again describing the kind of appointment that is needed.

In response to recognizing these disadvantages, the inventors have conceived and reduced to practice a software and/or hardware facility for using machine learning techniques to route consumer interactions from an automated mode of communication to a second mode of communication (“the facility”). In some embodiments, the facility routes such interactions from an automated mode of communication—i.e., with an automated agent—to a human mode of communication—i.e., with a human agent.

In some embodiments, the facility monitors each consumer's natural language interactions with an automated agent or other automated system. These can include, for example: text chat, via SMS or a dedicated app; email exchanges; voice conversation, via an audio and/or video connection; etc. Where the interactions are via voice, the facility performs automatic natural language transcription to transform the consumer's side of the interaction from voice into text.

In some embodiments, the facility subjects the text of the natural language exchange to a machine learning model to classify the consumer's intent. For example, the machine learning model may determine that a consumer's intent is to discover how long it takes to obtain the result of a particular medical test. The facility then determines whether the intent inferred by the machine learning model is well-suited to a human agent. If so, the facility prompts the consumer about interacting with a human agent. If the consumer chooses to do so, the facility applies a routing engine to select an appropriate category of human agent. For example, for the intent of discovering how long it takes to obtain a particular medical test result, the routing engine may select a “medical assistant” category of human agent.

The routing engine communicates with one or more backend systems-such as an IVR system, for example—to obtain current status information for this human agent category, such as (1) possible modes, which can include voice and text, and (2) availability information for each mode, such as number of unoccupied agents, estimated wait time, average text chat latency, etc. In various embodiments, the routing engine (1) surfaces the details of the live agent communication options to the consumer, who can then choose to proceed with whichever communication mode works best for them, or (2) automatically selects a mode, such as based on estimated wait time. The facility then communicates with the appropriate backend system to perform handoff of the consumer to a human agent in the selected category, in the selected mode. In some embodiments, this handoff includes information about the interaction so far, which can include either or both of (1) some or all of the transcript of the interaction, and (2) additional information about the consumer, such as information extracted from an electronic medical record (“EMR”) entry maintained for the consumer. As the result of this handoff, a human agent in the selected category takes up the interaction with the consumer—such as by voice or by text chat—with access to the provided context information about the interaction.

By operating in some or all of the ways described above, the facility helps consumers decide that a human mode of communication is better-suited for addressing their concern, chooses an appropriate category of human agents, helps the consumer select the best mode for interacting with them, and assigns the interaction to a particular agent in the category with the context needed to be helpful with minimum reliance on the consumer to repeat information already given in the interaction.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by switching an interaction to a better-suited mode as early in the interaction as this is discernable, the facility causes less time to be spent on less well-suited modes, such that fewer computing and communication resources are expended overall. Also, by providing to the human agent fuller context on the first part of the interaction, the facility causes fewer computing and communication resources to be expended on repeating communications that occurred earlier in the interaction.

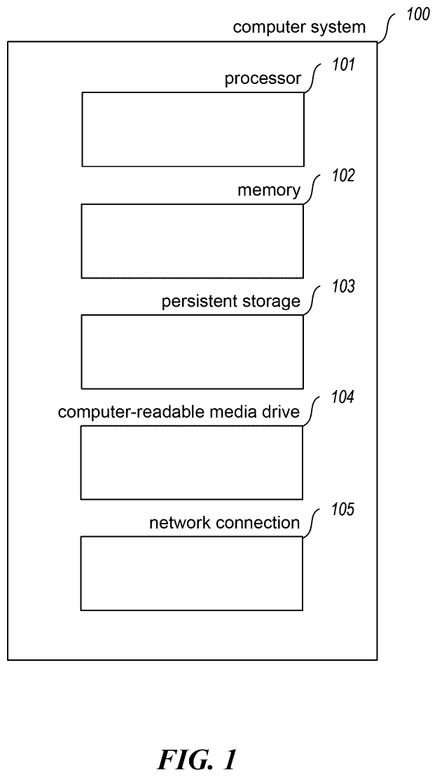

FIG. 1 is a block diagram showing some of the components typically incorporated in at least some of the computer systems and other devices on which the facility operates. In various embodiments, these computer systems and other devices 100 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 101 for executing computer programs and/or training or applying machine learning models, such as a CPU, GPU, TPU, NNP, FPGA, or ASIC; a computer memory 102 for storing programs and data while they are being used, including the facility and associated data, an operating system including a kernel, and device drivers; a persistent storage device 103, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

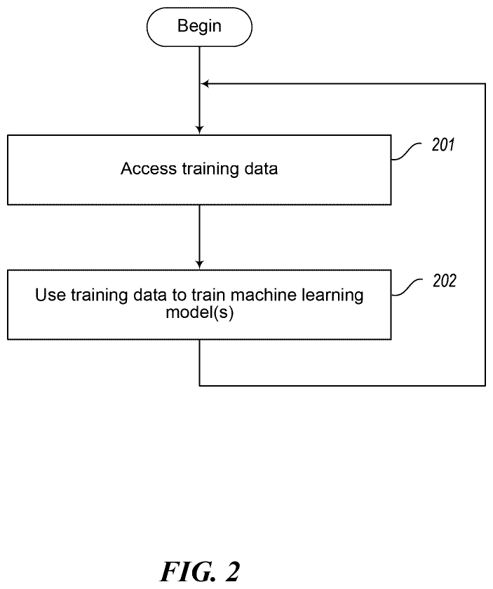

FIG. 2 is a flow diagram showing a process performed by the facility in some embodiments in order to train one or more machine learning models used by the facility. In some embodiments, the models trained by the facility include one that takes as an independent variable part or all of the transcript of a consumer interaction with an automated agent, and produces as a dependent variable a score or binary flag regarding the suitability of one or more modes of human communication for this interaction. In some embodiments, the models include a model that takes the same transcript as an independent variable, and produces as a dependent variable an intent inferred for the consumer from the interaction. In some embodiments, the models include a model that takes the same transcript as an independent variable, and produces as a dependent variable an entity inferred to have been referenced by the consumer in the interaction. In some embodiments, the models used by the facility include a model that takes the same transcript as an independent variable, and produces as a dependent variable a mode of communication inferred to be most appropriate for the interaction. In some embodiments, models used by the facility include a model that takes as an independent variable an intent determined for a consumer in an interaction, and produces as a dependent variable a category of human agent inferred to be most useful in participating in the interaction.

In act 201, the facility accesses training data for use in training one or more of the models used by the facility. In various embodiments, the training data includes the transcript of consumer interactions that have already occurred, in various embodiments including transcripts from interactions with an automated agent, transcripts from interactions with a human agent, or both. In some embodiments, the training data includes explicit or implicit indications by human agents that interactions that the human agent handled were well-suited to human modes of communication, and/or were well-suited to a particular category of human agent, and/or corresponded to a particular intent and/or a particular entity.

In act 202, the facility uses the training data to train one or more machine learning models used by the facility. In various embodiments, this training trains models of types such as long short-term memory networks (“LSTMs”) described by Sepp Hochreiter, Jürgen Schmidhuber, Long Short-Term Memory, Neural Comput 1997, 9 (8): 1735-1780, available at doi.org/10.1162/neco.1997.9.8.1735 or neural networks of other types; bidirectional encoder representations from transformers (“BERT”) described by Devlin, J., Chang, M., Lee, K., & Toutanova, K. (2019), BERT: Pre-training of Deep Bidirectional Transformers for Language Understanding, available at arxiv.org/abs/1810.04805 and/or dual intent and entity transformer (“DIET”) described by Mandy Mantha, Introducing DIET: state-of-the-art architecture that outperforms fine-tuning BERT and is 6× faster to train, Mar. 9, 2020, available at rasa.com/blog/introducing-dual-intent-and-entity-transformer-diet-state-of-the-art-performance-on-a-lightweight-architecture or other transformer deep learning models; GPT-3 described by Brown, Tom, et al, “Language models are few-shot learners,” Advances in neural information processing systems 33 (2020): 1877-1901, available at arxiv.org/abs/2005.14165, or other large language models, etc. Each of the documents identified above is hereby incorporated by reference in its entirety. In cases where a document incorporated by reference conflicts with the direct contents of this application, the direct contents of this application control. After act 202, the facility continues in act 201 to retrain these models at a later time using updated training data.

Those skilled in the art will appreciate that the acts shown in FIG. 2 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

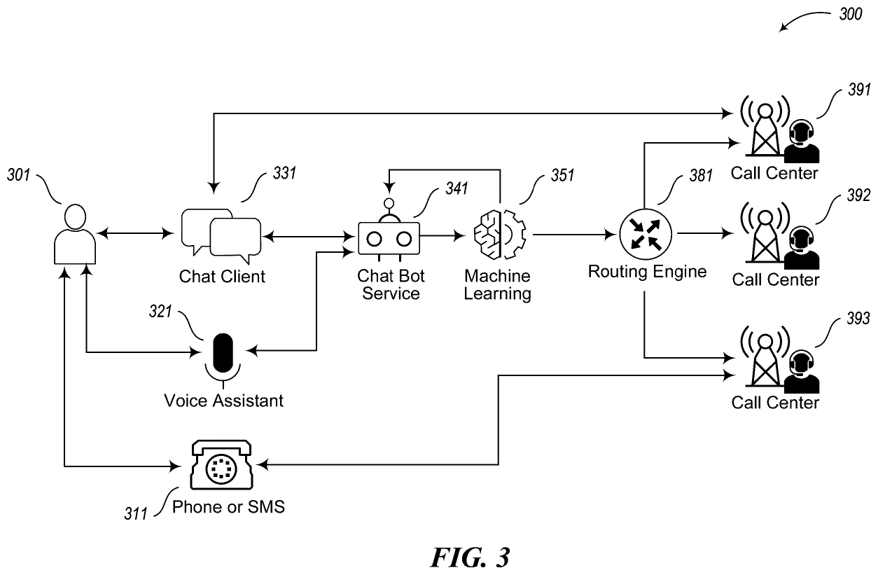

FIG. 3 is a data flow diagram showing the operation of the facility in some embodiments to route consumer interactions between communication modes. A consumer 301 initiates an interaction with an organization via its chat bot service 341, which conducts automatic communication with the consumer via various modes, such as a chat client 331 for textual communication, or a voice assistant 321 for voice communication. In various embodiments, the facility makes use of an off-the-shelf chat bot service, such as Sendbird, Twilio, TokBox, or Microsoft Bot Framework. The chat bot service passes transcripts of the interaction to one or more machine learning models 351. The machine learning models perform inference against the transcripts to determine whether they reflect interactions that are well-suited to human communication. In some embodiments, the machine learning models also infer one or more of expressed intent, expressed entity, and appropriate human agent category. In some embodiments, the facility uses a routing table or routing rules in another form to map from intent and/or entity inferred by the machine learning models to communication mode and/or human agent category. In various embodiments, these routing rules are determined by human editors, and/or determined empirically by the facility.

Where the facility determines that an interaction is well-suited to human communication, a routing engine 381 accesses status information for one or more call centers 391-393 or other services coordinating and/or monitoring the work of human agents, and particularly for human agents in the human agent category determined to be well-suited to the interaction. In various embodiments, the status information includes the number of agents in this category that are working, their present volume of work, their present availability for work, wait times to be able to speak to a human agent via voice, average latency of human agent in responding to textual lines of chat with other consumers, etc. On the basis of this status information, the routing engine either automatically selects a call center and human mode of communication to which to transition the interaction, or presents available options to the consumer, in some cases with some or all of the received status information. The facility then transitions the interaction to the selected mode of communication and human agent category. In various embodiments, this involves shifting a textual chat session to a human agent in the selected category; transferring a voice call to a human agent in the selected category; collecting a callback number from the consumer that is used to put the consumer in touch with a human agent when the human agent becomes available; etc. In some embodiments, the routing engine routes certain interactions to other forms of communication, such as self-service mechanisms such as forms or wizards with which the consumer can interact via typing or voice without the involvement of any human agents.

The consumer thereafter interacts 311—such as via phone or text messaging—with the human agent or other resource to which the consumer's interaction was routed by the facility. In some embodiments, the routing involves passing context information about the interaction for use in its subsequent servicing. For example, in some embodiments, the human agent to whom the interaction is routed sees the earlier exchange of textual messages between the consumer and the automated agent, in some cases as part of the same transcript that textual messages between the human agent and the consumer are shown after they're sent.

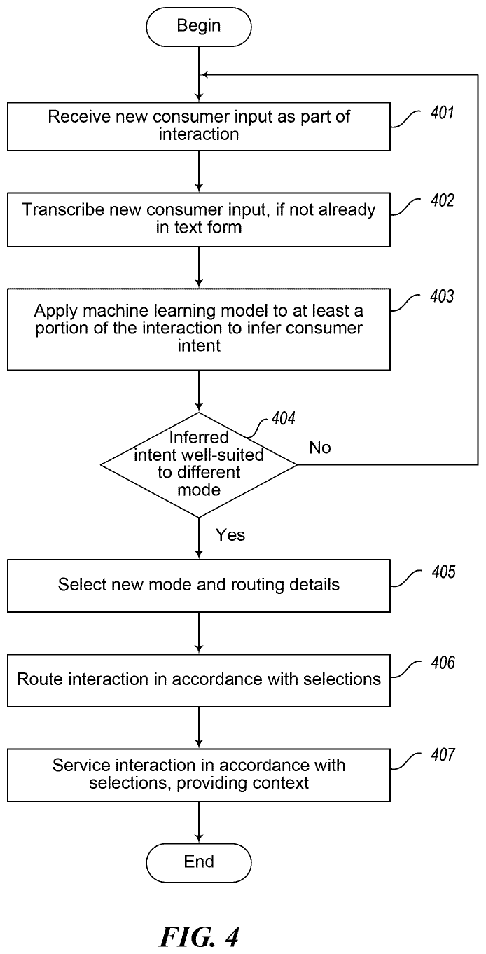

FIG. 4 is a flow diagram showing a process performed by the facility in some embodiments to route consumer interactions between communication modes. In act 401, the facility receives new consumer input as part of an interaction, such as a new line of text, or a new spoken sentence. In act 402, the facility transcribes the new consumer input received in act 401, if it is not already in textual form. In act 403, the facility applies one or more machine learning models to at least a portion of the textual version of the interaction to infer consumer intent, and in some embodiments, an entity referenced in the interaction. In various embodiments, various portions of the interaction are submitted to the machine learning model in act 403, many of which contain at least the new consumer input received in the last iteration of act 401.

In some embodiments, the facility infers an intent common among medical patients, including such intents as ambiguous pain symptoms, ambiguous feelings, general check-in, and other ambiguous diagnosis intents; respiratory symptoms, upper respiratory symptoms, musculoskeletal symptoms, feet-related problem, miscellaneous identified symptoms, dermatological problem, and other identified symptom intents; chronic cardiovascular/diabetes condition, medication, nutrition, joint procedure, patient-initiated care, miscellaneous chronic condition, surgical procedure, and other condition management intents; test result, blood test, imaging exam, and other tests/exams intents; medical referral and other clinical decision-making referral intents; miscellaneous paperwork, insurance, general paperwork, forms, and other paperwork intents; scheduling appointment, scheduling uncompleted calls, and other scheduling intents; administrative referral, family referral, and other referral intents; and prescription administrative problem, refill coordination, and other prescription intents.

In act 404, if the application of the models in act 403 produces an inference that the interaction is well-suited to a different mode of communication, then the facility continues in act 405, else the facility continues in act 401 to receive the next consumer input as part of the interaction in the same mode of communication. In act 405, the facility selects a new mode of communication, as well as details used to route the interaction to a particular resource to be handled using the new mode of communication. In act 406, the facility routes the interaction in accordance with the selections of act 405. In act 407, the facility services the interaction in accordance with the selections of act 405, in some embodiments providing context about the interaction, such as the textual transcript of some or all of the interaction up to this point. After act 407, this process concludes.

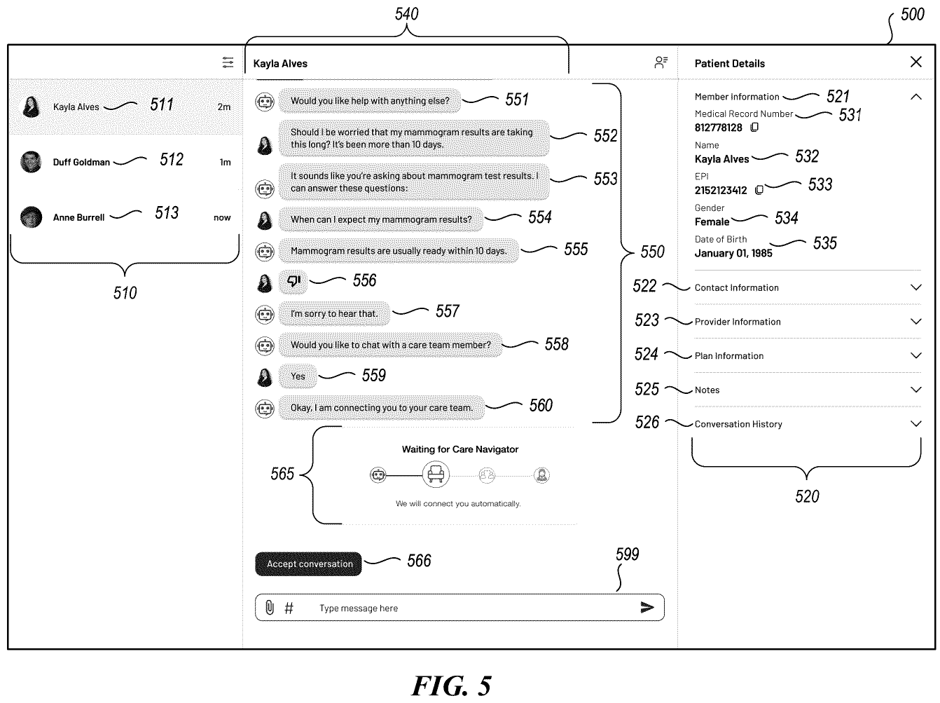

FIG. 5 is a display diagram showing sample contents of a first display presented by the facility in some embodiments to a human agent to whom an interaction is routed by the facility. The display 500 includes a list 510 of active textual interactions this human agent is having with consumers 511-513. Because consumer 511 has been selected by the human agent, in list 510 transcript 540 shows the text messages exchanged with the consumer as part of the interaction. In particular, text messages 550 were exchanged between the consumer and the automated agent. For example, the automated agent sent text messages 551, 553, 555, 557, 558 and 560, while the consumer sent text messages 552, 554, 556, and 559. Subsequently, the automated agent and/or another part of the facility such as the routing engine sent the consumer indication 565 that the connection is being transitioned to a human agent. The display also contains a control 566 that the human agent can activate in order to enter the interaction, as well as a control 599 that the human agent can use in order to send textual messages to the consumer as part of the interaction.

The display also includes contextual information 520 that goes beyond the present interaction, including sections about member information 521, member contact information 522, medical provider information 523, medical insurance plan information 524, patient notes 525, and history 526 of past interactions with the consumer. The member information section 521 is expanded, showing constituent details medical record number 531, consumer name 532, EPI identifier 533, sex 534, and birthdate 535. The human agent can similarly expand the other sections by selecting them.

While FIG. 5 and each of the display diagrams discussed below show a display whose formatting, organization, informational density, etc., is best suited to certain types of display devices, those skilled in the art will appreciate that actual displays presented by the facility may differ from those shown, in that they may be optimized for particular other display devices, or have shown visual elements omitted, visual elements not shown included, visual elements reorganized, reformatted, revisualized, or shown at different levels of magnification, etc.

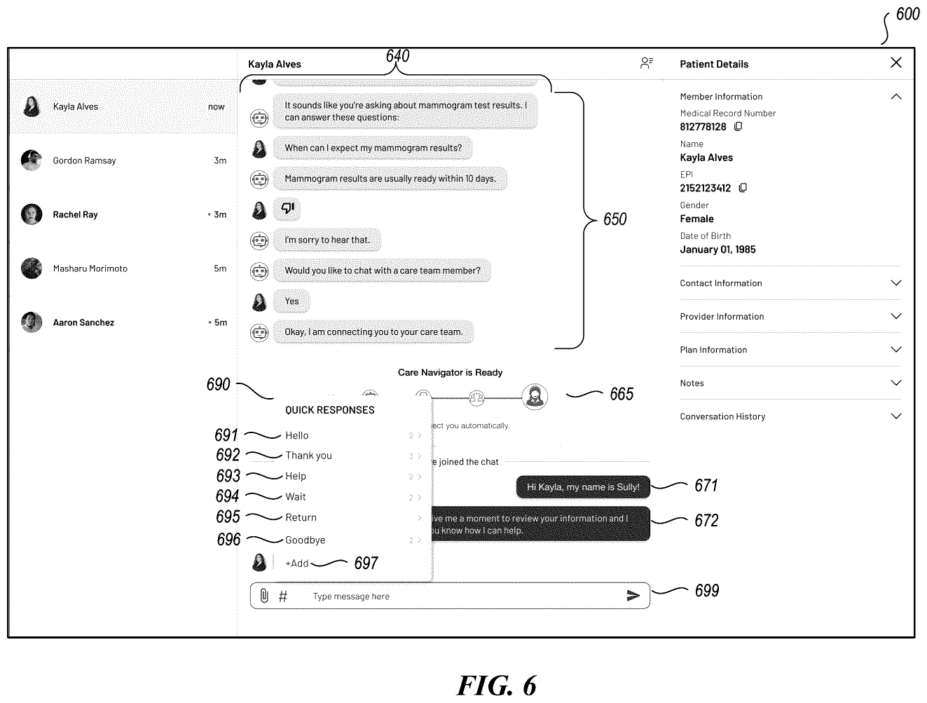

FIG. 6 is a display diagram showing sample contents of a second display presented by the facility in some embodiments to a human agent to whom an interaction is routed by the facility. The display 600 shows text messages 671 and 672 sent as part of the interaction from the human agent to the consumer. It also shows a quick response menu 690 displayed to the human agent, in which the human agent can select any of the quick response titles 691-696 in order for the content of the corresponding quick response to be sent as a textual message from the human agent to the consumer. For example, in some embodiments, the human agent can send message 671 by selecting quick response title 691. The menu also includes an Add control 697 that the human agent can select in order to add a new quick response, such as by typing its title and its content.

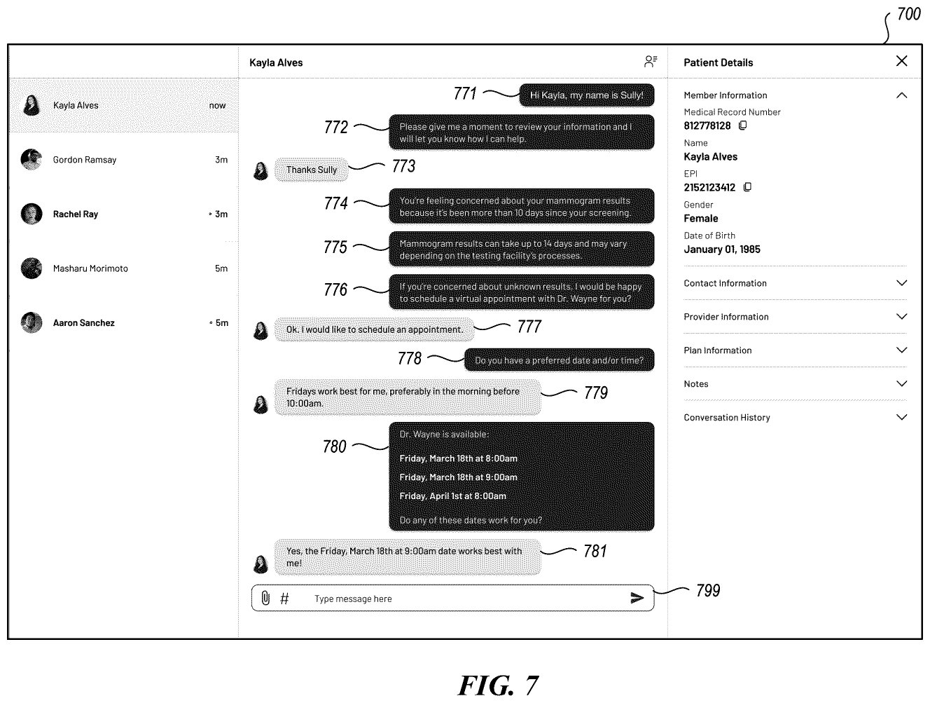

FIG. 7 is a display diagram showing sample contents of a third display presented by the facility in some embodiments to a human agent to whom an interaction is routed by the facility. Display 700 shows additional textual messages 773-781 exchanged between the human agent and the consumer.

In some embodiments, the facility presents displays like those shown in FIGS. 5-7 to a human agent engaged in human voice interaction with one or more consumers.

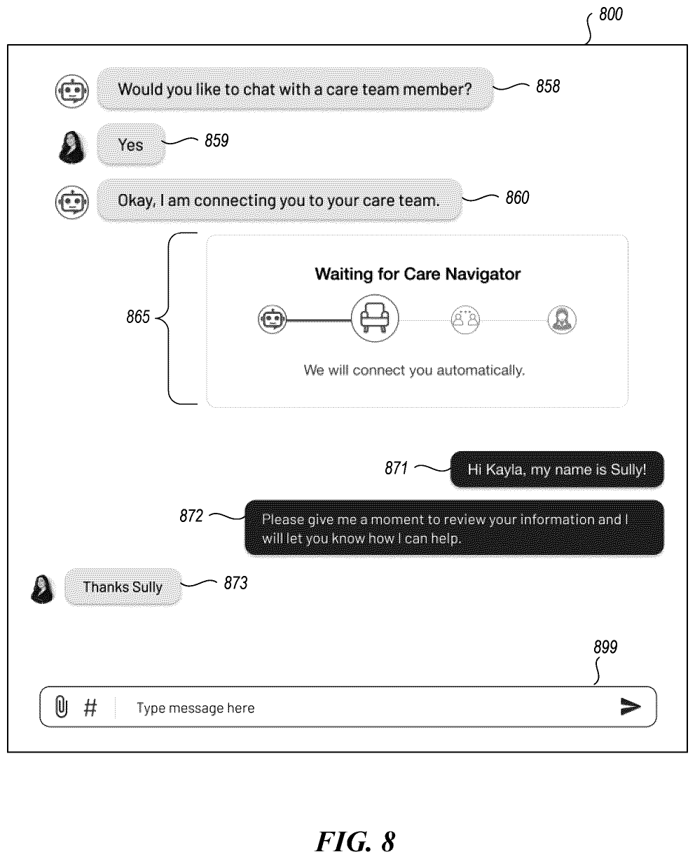

FIG. 8 is a display diagram showing a sample display presented by the facility to a consumer in some embodiments. The display 800 corresponds to displays 500, 600, and 700 shown in FIGS. 5-7, and in particular shows the consumer's side of the interaction shown from the human agent's perspective in those figures. The display 800 contains text messages 858-860 exchanged between the consumer and the automated agent; the transition message 865, and then text messages 871-873 exchanged between the consumer and the human agent. By comparing FIG. 8 to FIG. 6, it can be seen that these messages correspond to the those displayed by the facility to the human agent.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system, comprising:
receiving a request for live interaction from a user;
in response to receiving the request, causing an automatic live interaction to be conducted with the user in which one or more first messages are received from the user, and one or more second messages are sent to the user;
periodically during the automatic live interaction:
using an up-to-date textual transcript for the automatic live interaction to assess whether the live interaction is one well-suited to a human live interaction;
in response to determining that the live interaction is well-suited to a human live interaction:
causing to be initiated between the user and a human agent a human live interaction in place of the automatic live interaction;
in connection with causing the initiating, causing to be presented to the human agent text corresponding to at least some of the first messages and at least some of the second messages,
wherein the assessing comprises:
applying a trained machine learning model to at least a portion of up-to-date textual transcript for the automatic live interaction to obtain a predicted intent of the user in the automatic live interaction; and
determining whether the predicted intent is one well-suited to a human live interaction.

2. The method of claim 1 wherein the automatic live interaction is via text.

3. The method of claim 1 wherein the automatic live interaction is via voice, the method further comprising causing the first messages received from the user in voice form to be automatically transformed into text form.

4. (canceled)

5. The method of claim 41, further comprising:
accessing training data representing live interaction transcripts for each of which an intent has been determined; and
using the accessed training data to train the machine learning model that is applied.

6. The method of claim 1 wherein the trained machine learning model is of one or more of the following machine learning model types:
long short-term memory network;
neural network;
bidirectional encoder representations from transformers;
dual intent and entity transformer;
transformer deep learning model;
GPT-2; or
large language model.

7. The method of claim 1 wherein applying the trained machine learning model also obtains a predicted entity referenced by the user in the automatic live interaction,
and wherein the determination is further based on the predicted entity.

8. The method of claim 1, further comprising:
in response to determining that the live interaction is well-suited to a human live interaction:
selecting, based on the textual transcript for the automatic live interaction, one of a plurality of human agent categories as best-suited to take over the live interaction,
and wherein the initiated human live interaction is initiated with a human agent in the selected human agent category.

9. The method of claim 1, further comprising:
in response to determining that the live interaction is well-suited to a human live interaction:
causing to be presented to the human agent text corresponding to at least some of the first messages and at least some of the second messages.

10. The method of claim 9 wherein the causing text presentation causes the text to be presented in a first display location,
and wherein the human live interaction includes one or more third messages that are received from the user, and one or more fourth message originated by the human agent that are sent to the user,
the method further comprising causing to be presented to the human agent text corresponding to at least some of the third messages and at least some of the fourth messages, in a second display location adjacent to the first display location.

11. The method of claim 9, the method further comprising causing to be presented to the human agent information about the user that is not related to the live interaction.

12. One or more instances of computer-readable media collectively having contents configured to cause a computing system to perform a method, none of the one or more instances of computer-readable media constituting a signal per se, the method comprising:
receiving a request for live interaction from a user;
in response to receiving the request, causing an automatic live interaction to be conducted with the user in which one or more first messages are received from the user, and one or more second messages are sent to the user;
in place of the automatic live interaction, causing to be initiated a human live interaction between the user and a human agent; and
in connection with causing the initiating, causing to be presented to the human agent text corresponding to at least some of the first messages and at least some of the second messages, wherein the human live interaction is initiated in response to determining that the live interaction is well-suited to a human live interaction based upon using an up-to-date textual transcript for the automatic live interaction to assess whether the live interaction is one well-suited to a human live interaction,
wherein the assessing comprises:
applying a trained machine learning model to at least a portion of up-to-date textual transcript for the automatic live interaction to obtain a predicted intent of the user in the automatic live interaction; and
determining whether the predicted intent is one well-suited to a human live interaction.

13. The one or more instances of computer-readable media of claim 12 wherein the automatic live interaction is a voice interaction, the method further comprising:
causing the first messages to be automatically transcribed from voice form to text form to produce the presented text corresponding to at least some of the first messages.

14. The one or more instances of computer-readable media of claim 12 wherein the causing causes the text to be presented in a first display location,
and wherein the human live interaction includes one or more third messages that are received from the user, and one or more fourth message originated by the human agent that are sent to the user,
the method further comprising causing to be presented to the human agent text corresponding to at least some of the third messages and at least some of the fourth messages, in a second display location adjacent to the first display location.

15. The one or more instances of computer-readable media of claim 12, the method further comprising causing to be presented to the human agent information about the user that is not related to the live interaction.

16. The one or more instances of computer-readable media of claim 15, the method further comprising causing the presented information about the user that is not related to the live interaction to be retrieved from an EMR record corresponding to the user.

17. One or more instances of computer-readable media collectively storing a data structure adapted for use on behalf of an organization providing human agents in each of a plurality of human agent categories, none of the one or more instances of computer-readable media constituting a signal per se, the data structure comprising:
first information for selecting, for a textual transcript of an interaction between the user and an agent participating in the interaction on behalf of an organization, one of the plurality of human agent categories as best-suited to take over the interaction,
such that the first information is usable to perform selection of one of the plurality of human agent categories as best-suited to take over a particular interaction based on the particular interaction's textual transcript.

18. The one or more instances of computer-readable media of claim 17 wherein the first information comprises a plurality of entries, each entry comprising:
second information mapping from one possible intent that may be determined to be expressed by a user in a textual transcript of an interaction between the user and an agent participating in the interaction on behalf of any organization to one of the plurality of human agent categories.

19. The one or more instances of computer-readable media of claim 17 wherein the first information comprises a trained machine learning model that predicts the human agent category best-suited to take over a particular interaction based on the particular interaction's textual transcript.

20. The one or more instances of computer-readable media of claim 17 wherein the data structure further comprises:
for each of the plurality of human agent categories, second information specifying how to initiate a human live interaction with a human agent in the human agent category,
such that the second information is usable to initiate a human live interaction with a human agent in the human agent category selected for a particular interaction.
